(19) United States
(12) Patent Application Publication
(10) Pub. No.: US 2021/0353925 A1
(43) Pub. Date: Nov. 18, 2021

(54) REMOVABLE PARTIAL ARM RESTRAINT

(71) Applicant: Warner Weseman, Gainesille, FL (US)

(72) Inventor: Warner Weseman, Gainesille, FL (US)

(21) Appl. No.: 15/930,813

(22) Filed: May 13, 2020

(51) Int. Cl. A61M 39/02; A61G 13/12

(52) U.S. Cl. CPC A61M 39/0208; A61M 2039/0229; A61M 2039/0223; A61G 13/1235

(57) ABSTRACT

The present invention is an arm restraint used in dialysis primarily when the fistula is located at or near the elbow. The restraint supports the arm and allows a limited amount of movement of the restrained arm to mitigate discomfort during a dialysis procedure, while simultaneously allowing unrestricted blood flow in the supported arm. The restraint includes an adjustable fastener that allows the restraint to be attached to the upper arm in a loose and adjustable manner. The restraint includes a cover at its lower end that allows a limited degree of arm movement. It also includes an open upper area that enables access to the arm for placement and adjustment of dialysis syringes and associated tubing.

### FIELD OF THE INVENTION

The field of the invention relates generally to medical apparatus, more specifically to apparatus used in kidney dialysis procedures, and still more specifically to arm restraints and supports utilized during dialysis operations.

### BACKGROUND OF THE INVENTION

Kidney dialysis procedure are a well-known and common method to mitigate the symptoms found in kidneys afflicted with kidney disease. Millions of patients have their lives improved by undergoing dialysis procedures.

Unfortunately, dialysis is a process that that is both time consuming as well as at least uncomfortable and at worst painful. Patients usually undergo dialysis two or three times per week with each procedure lasting three to four hours. During the procedure, syringe type needles are inserted into a patient's arm in a fistula and connected by tubing to the dialysis machine. The blood flows from the body into the dialysis machine to be filtered and then back into the body. A fistula is defined as an abnormal passage that leads from an abscess or hollow organ or part to the body surface or from one hollow organ or part to another and that may be surgically created to enlarge and surgically join a vein and an artery permit passage of fluids or secretions.

While effective in purifying the blood, the described setup creates several problems. First, the patient cannot move his arm from the extended position that is necessary to allow the needles to access the correct area of the arm. After some time, the patient experiences some discomfort and will shift the position of the arm too much causing the needles to be dislodged from the fistula and lodge in the arm itself. While it has been found that some slight arm movements are acceptable, often the patient shifts his arm too much. This movement often occurs unconsciously. This dislodged condition, called infiltration, is painful and can create bruising that lasts long after the procedure itself is completed. The discomfort is increased as the syringes must be reattached to the fistula.

In addition, the problem of dislodgement is compounded by the frequent adjustment of the tubing and needles during the dialysis procedure. The dialysis machine is equipped with alarms that signal the need for adjustments in either the machine itself or the needles and connected tubing. Access to the needles and tubing is necessary in order to perform adjustments to move the dialysis out of the alarm condition.

Finally, for dialysis patients, there must be unrestricted blood flow in the target arm before, during and after administering the dialysis procedure on a 24/7 basis. This includes prohibitions on picking up items weighing 20 pounds or more, drawing blood, and taking blood pressure readings. This requirement precludes the use of tight straps as well as shots, transfusions and muscle contractions.

The prior art includes arm supports designed to hold an arm in position during dialysis. U.S. Pat. No. 4,489,716 to Blackwood discloses a brace designed to prevent hyperextension of the elbow. It includes flexible hook and loop straps such as Velcro® designed to hold the arm firmly in place. It provides no measured freedom of movement of the arm and the straps hinder access to the needles and tubing to make the adjustments discussed above. In addition, the straps would restrict blood flow in the supported arm. U.S. Pat. No. 5,746,705 to Sheppard discloses a modified arm sling comprising a first elongated member joined to a second elongated member. The structure of the sling prevents access to the site of a fistula especially fistula sites at or near the elbow. Chinese patent CN208911087U to Yan Xiaohui, et al. teaches an arm support having a cylindrical shape and a blood pressure cuff at the upper end. However, it lacks a partial bottom end restraint and an upper end fastener to hold it to the arm. It would likely prevent any type of movement of the arm in general and the blood pressure cuff prevents its use during dialysis as it would restrict blood flow

Chinese patent CN20952950U to Li Yanping discloses a temperature-controlled arm support. It includes air bags on either side of a tube, and Velcro® straps at each end that hold the bags to the tube. The straps also hold the arm in place and allow no freedom of movement for the arm as well as interfering with adjustment of the dialysis syringes and tubing. Additionally, the straps restrict blood flow in the supported arm. Similarly, Chinese patent CN203043088 to Li Yajuan et al. is directed to an arched limb fixator made from a thermoplastic material and equipped with bands to hold the arm in firmly in place. Again, in the limb fixator, there is no provision for any measured freedom of movement of the arm, the straps interfere with adjusting the syringes and tubing, and the bands serve to restrict blood flow.

Japanese Patent No. JP20022172164A to Nobukawa teaches an arm protector to prevent dislodgement of the dialysis needles from a patient's arm. It includes a bottom rest plate with an arcuate cover with the arm extending through the entire device so the wrist and hand extend through end of the protector. The cover prevents access to the dialysis needles and tubing during dialysis and allows excessive freedom of movement of the patient's arm creating the potential of initiating numerous alarms and pauses for syringe and tubing adjustments.

The prior art contains numerous examples of arm supports that may be used in a dialysis situation. However, nothing in the prior art provides for a restraint that allows for a measured or limited amount of movement while still preventing both dislodgement of the needles and restriction of blood flow and allows access to the needles to adjust them when the dialysis machine is in alarm mode.

### SUMMARY

The present invention broadly comprises a partial arm movement restraint that comprises an elongated body such that the elongated body includes at least: a nonplanar bottom surface; two opposing sides; an open upper end; an open lower end; and, a rigid cover surrounding the lower open end. The invention also includes an adjustable fastening device attached to the upper open end to attach the movement restraint device preferably in a loose and adjustable manner to a portion of an upper arm; wherein an upper portion of the elongated body is open. By loosely is meant that the adjustable fastening device is attached to the upper arm such that is holds the restraint to the upper arm but does not restrict blood flow in the target arm. In a one embodiment, the rigid cover is a ring. In another embodiment, the rigid cover is integral with and surrounds the open lower end of the elongated body.

One object of the invention is to minimize patient discomfort during dialysis by allowing a limited degree of movement of a patient's arm while still preventing or reducing the number of dialysis needle adjustments or dislodgements during dialysis.

A second object of the invention is to allow easy access to the dialysis needles and tubing by medical personnel.

A third object of the invention is to provide a support that does not restrict blood flow in the arm receiving the dialysis treatment.

An additional object of the invention is to provide a limb restraint that is easy to put on and remove.

A further object of the invention is to supply a restraint suitable for used when the fistula is located at or near the elbow.

### BRIEF DESCRIPTION OF THE SEVERAL VIEWS OF THE DRAWINGS

The nature and mode of the operation of the present invention will now be more fully described in the following detailed description of the invention taken with the accompanying drawing Figures, in which:

FIG. 1 is a top view of one embodiment of removable partial movement arm restraint of the present invention;

FIG. 2 is a view of the present invention from the open upper end;

FIG. 3 is a top perspective view of the partial movement restraint of the present invention;

FIG. 4 is a top perspective view depicting an embodiment in which the arm restraint of the present invention is supporting a lower arm;

FIG. 5 is a view from the open lower end showing the hand and wrist extending out of the lower end; and,

FIG. 6 is the same view as in FIG. 5 showing the syringe needles placed in the lower arm.

### DETAILED DESCRIPTION OF EMBODIMENTS OF THE INVENTION

At the outset, it should be appreciated that like drawing numbers on different drawing views identify identical structural elements of the invention. It also should be appreciated that figure proportions and angles are not always to scale in order to clearly portray the attributes of the present invention.

While the present invention is described with respect to what is presently considered to be the preferred embodiments, it is understood that the invention is not limited to the disclosed embodiments. The present invention is intended to cover various modifications and equivalent arrangements included within the spirit and scope of the appended claims.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. It should be appreciated that the term “substantially” is synonymous with terms such as “nearly”, “very nearly”, “about”, “approximately”, “around”, “bordering on”, “close to”, “essentially”, “in the neighborhood of”, “in the vicinity of”, etc., and such terms may be used interchangeably as appearing in the specification and claims. It should be appreciated that the term “proximate” is synonymous with terms such as “nearby”, “close”, “adjacent”, “neighboring”, “immediate”, “adjoining”, etc., and such terms may be used interchangeably as appearing in the specification and claims. Although any methods, devices or materials similar or equivalent to those described herein can be used in the practice or testing of the invention, the preferred methods, devices, and materials are now described.

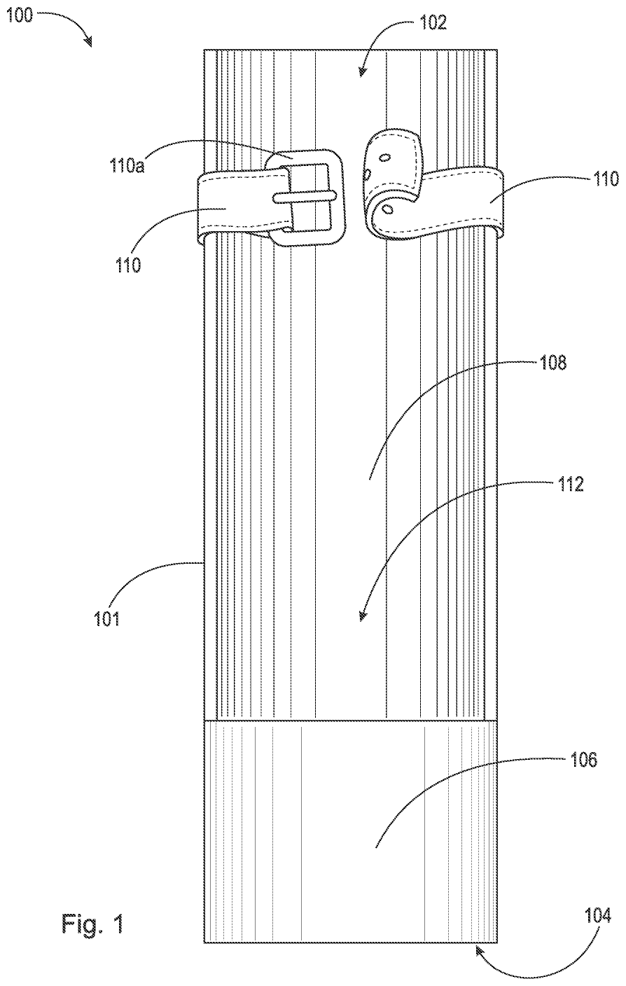

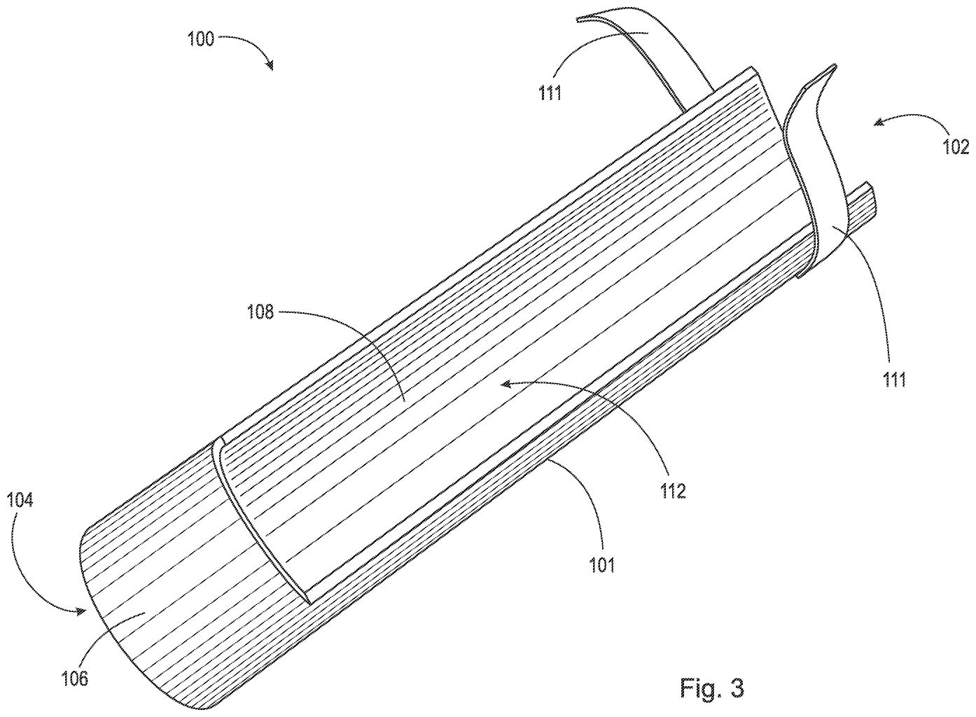

Adverting to the drawings, FIG. 1 is a top view of one embodiment of removable partial movement arm restraint 100 (“restraint 100”) of the present invention. Restraint 100 includes elongated body 101 (“body 101”) which comprises open upper end 102 and open lower end 104. By open is meant, each end is at least partially surrounded by the upper or lower edge, respectively, of upper end 102 and lower end 104 but the space within the respective edges can be seen through and an object can extend through the respective ends. Rigid cover 106 is located at or near lower end 104 and is attached to body 101. In one embodiment, cover 106 is integral with body 101 as seen in FIG. 3 below. In an alternate embodiment, cover 106 may be attached to body 101. As shown, cover 106 is arched or circular, but persons of skill in the art will recognize that cover 106 may have other shapes, such as, but not limited to, a v shape or oblong shape as long as there is sufficient space under cover to allow a limited degree of movement of the arm. By limited is meant that the arm may be moved slightly but not enough to create an alarm condition in the dialysis machine or associated tubing and syringes.

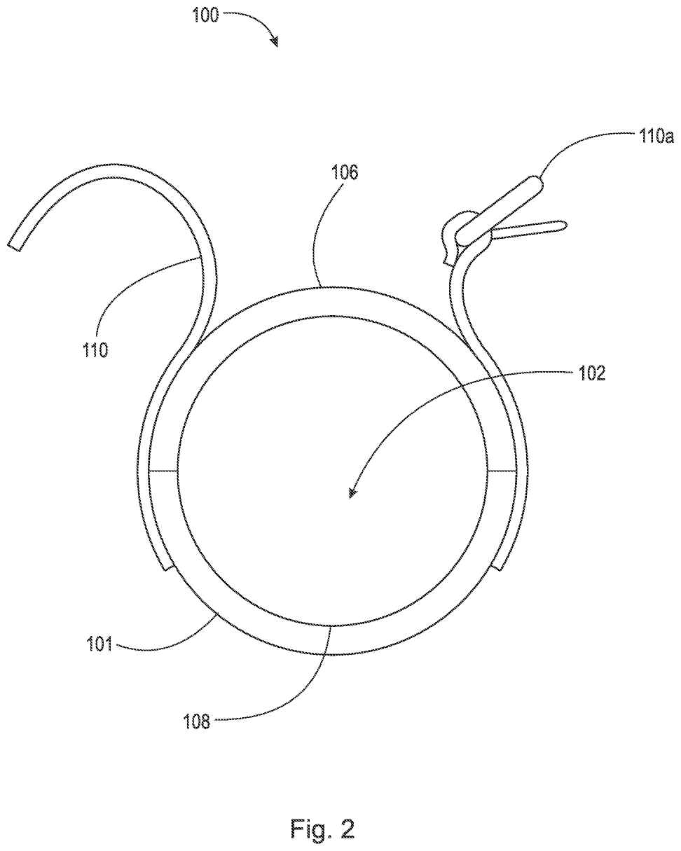

Bottom 108 is nonplanar, i.e., not flat, which has the advantage being more comfortable than a flat surface when supporting a patient's arm. As shown in the figures, bottom 108 has a cylindrical surface. In alternate embodiment, bottom 108 may be shaped like a trough or have a V-shape. In one embodiment, padding (not shown) may be applied to bottom 108. Strap 110 is located near upper end 102 and forms the attachment of restraint 100 to the patient's upper arm. In the embodiment shown, adjustable strap 110 is similar to a belt buckle assembly and includes buckle 110a. Open top area 112 (“area 112”) is defined by the sides of body 101 and cover 106. As described below, area 112 is large enough to enable the syringe needles and tubing to be easily placed in the dialysis patient's arm and allows easy access to adjust the needles and tubing in alarm situations. FIG. 2 is a view of restraint 100 from upper end 102 showing cover 106 at lower end 104 and strap 110.

FIG. 3 is a top perspective view of restraint 100 showing the integral connection of cover 106 to body 101. By integral is meant that the component parts are formed together as a single unit. Also depicted is an alternate embodiment to strap 110 as it is replaced by a hook and loop fastener 111 that is adjustable and like strap 110, is adjustable to fit loosely around upper arm UA so as not to impede blood flow in the target arm from which the dialysis procedure is taking place. Fastener 111 may be fabricated using material such as VELCRO®.

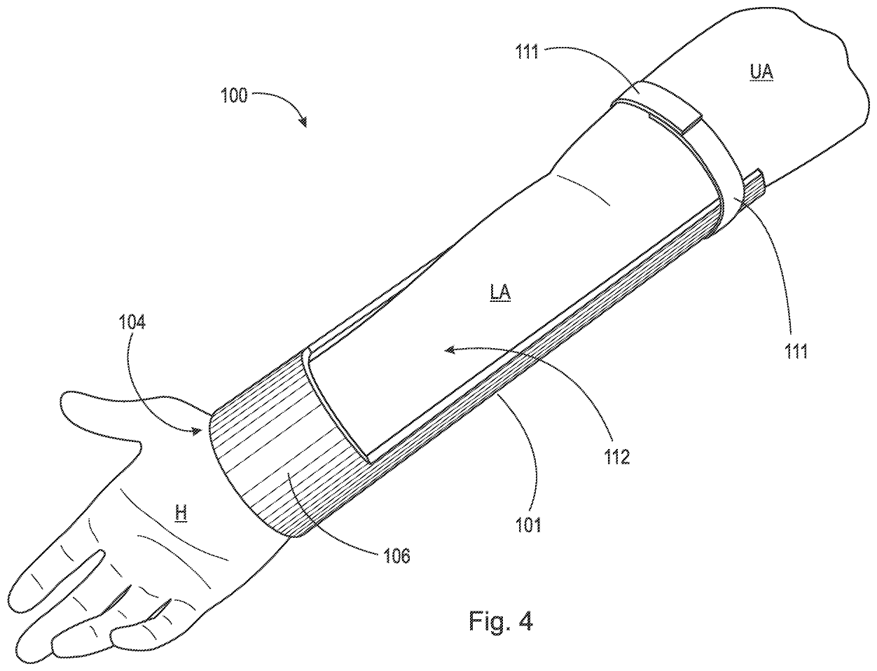

FIG. 4 is a top perspective view depicting an embodiment in which restraint 100 is supporting lower arm LA. Hand H is shown extending through lower end 104 with strap 111 wrapped loosely around upper arm UA just above the elbow. In an alternate embodiment (not shown) straps 110 or 111 may be wrapped around lower arm LA. It can be seen that lower arm LA is very exposed in open area 112 providing easy access for placement of the dialysis needles and tubing.

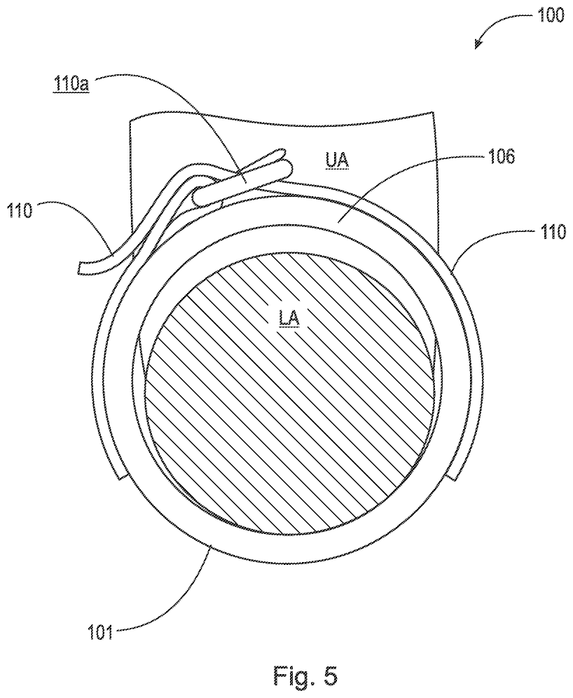

FIG. 5 is a view from open lower end 104 showing lower arm LA extending out of lower end 104. It can be seen that lower arm LA is partially restrained by cover 106 and body 101 of restraint 100, but there is still sufficient space between lower arm LA and the bottom surface of cover 106 to not restrict blood flow and still allow a limited amount of movement not only of lower arm LA but also of the wrist and hand H.

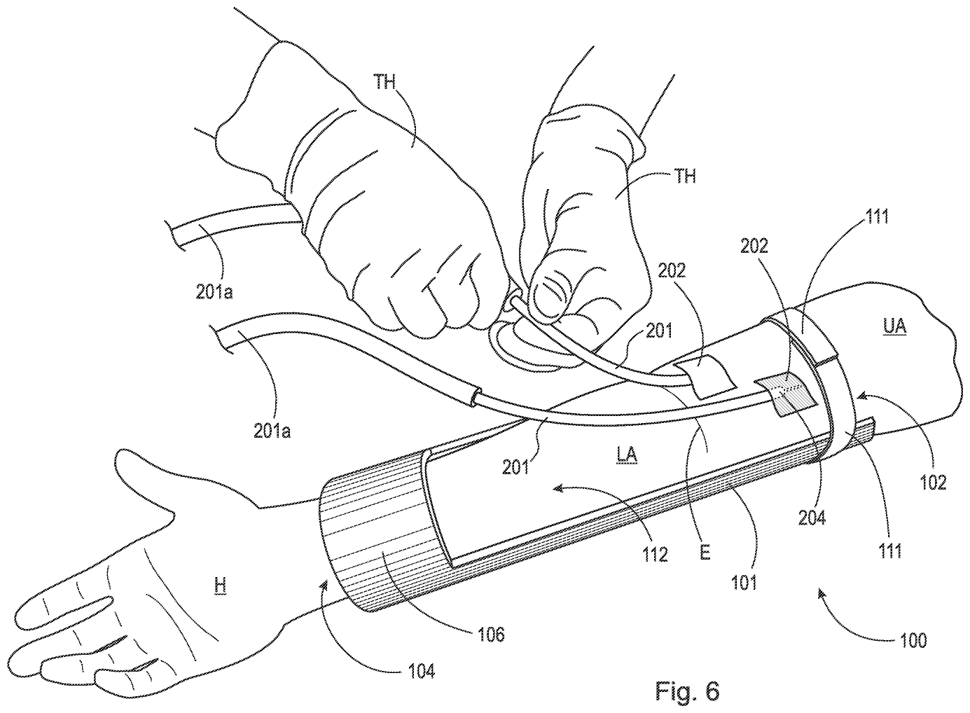

FIG. 6 is the same view as in FIG. 4 showing the syringe needles 204 placed in upper arm UA located at or near the elbow E and covered by bandages 202. Also seen are the hands TH of a technician demonstrating the accessibility of syringes 204 and tubing 201 to enable them to adjust their positions as required, especially in alarm situations. In the situation depicted in FIG. 6, tubing 201 is connected to a larger tubing 201a which is connected to the dialysis machine (not shown).

Preferably, syringe needles 204 with attached tubing 201 are inserted into upper arm UA at or near the elbow with lower arm LA placed into restraint 100. Restraint 100 is then loosely attached to upper arm UA using fastener 110 or an alternate suitable fastener, such as hook and loop fastener 111 to hold restraint 100 to upper arm UA. After restraint 100 is attached to upper arm UA, each of tubing 201 or 201a is attached to appropriate couplings on the dialysis machine (not shown). It can be seen from FIGS. 5 and 6 that because of the lack of straps around lower arm LA and the space between lower arm LA and cover 106, as well as open area 112, that restraint 100 provides the advantage of not restricting blood flow during dialysis as no restrictive devices such as bands or straps are placed around lower arm LA.

After the dialysis procedure is completed, to remove restraint 100, tubing 201 or 201a is first detached from the dialysis machine. Restraint 100 is then unfastened from upper arm UA after which the syringe needles 204 are removed from lower arm LA with tubing 201 still attached to syringes needles 204.

Thus, it is seen that the objects of the invention are efficiently obtained, although changes and modifications to the invention should be readily apparent to those having ordinary skill in the art, which changes would not depart from the spirit and scope of the invention as claimed.

I claim:

1. A partial arm movement restraint device comprising:
an elongated body wherein said elongated body includes:
a nonplanar bottom surface;
two opposing sides;
an open upper end;
an open lower end;
a rigid cover surrounding said lower open end; and,
an open area defined by said rigid cover and said two opposing sides; and, an adjustable fastening device attached to said upper open end to attach said movement restraint device to an upper portion of an arm.

2. The device of claim 1 wherein said adjustable fastening device is a belt.

3. The device of claim 1 wherein said adjustable fastening device is a strap.

4. The device of claim 3 wherein said adjustable fastening device is a hook and loop fastener.

5. The device of claim 1 wherein said nonplanar bottom surface is curved.

6. The device of claim 1 wherein said nonplanar bottom surface is trough shaped.

7. The device of claim 1 wherein said nonplanar bottom surface is v-shaped.

8. The device of claim 1 further comprising padding in contact with said nonplanar bottom surface.

9. The device of claim 1 wherein said rigid cover is removably attached to said open lower end.

10. The device of claim 1 wherein said rigid cover is fixedly attached to said open lower end.

11. The device of claim 1 wherein said rigid cover is integral with said open lower end.
